(19) United States
(12) Patent Application Publication
(10) Pub. No.: US 2003/0170774 A1
(43) Pub. Date: Sep. 11, 2003

(54) Fermentative process for obtaining natural aromatic, aliphatic and thiocarboxylic acids and microorganism therefor

(76) Inventors: Jurgen Rabenhorst, Hoxter (DE); Ian-Lucas Gatfield, Hoxter (DE); Jens-Michael Hilmer, Hoxter (DE)

Correspondence Address: PENDORF & CUTLIFF, P.O. Box 20445, Tampa, FL, 33622-0445, US

(21) Appl. No.: 10/377,505

(22) Filed: Feb. 28, 2003

Related U.S. Application Data
(62) Division of application No. 09/639,685, filed on Aug. 16, 2000.

(30) Foreign Application Priority Data
Aug. 25, 1999 (EP) 99116711.5

(51) Int. Cl. C12P 1/00; C12P 11/00

(52) U.S. Cl. 435/41

(57) ABSTRACT

The present invention relates to a process for the preparation of aliphatic, aromatic and thiocarboxylic acids, where cultures comprising bacteria of the genus Gluconobacter are used.

### FIELD OF THE INVENTION

The present invention relates to a biological process for the preparation of various carboxylic acids by the enzymatic oxidation of the corresponding alcohols with alcohol oxidase in very high yields. The alcohol oxidase is formed by bacteria of the genus Gluconobacter, preferably of the species Gluconobacter sp. HR 101 (DSM 12884). Thus, for example, benzoic acid is prepared from benzyl alcohol, butyric acid from n-butanol, isobutyric acid from isobutanol, isovaleric acid from isoamyl alcohol, 2-methylbutyric acid from 2-methylbutanol, 3-methylthiopropionic acid from 3-methylthiopropanol, phenylacetic acid from phenylethanol, propionic acid from propanol and cinnamic acid from cinnamyl alcohol.

### BACKGROUND OF THE INVENTION

In addition to the long-known process for the manufacture of vinegar by oxidizing ethanol to give acetic acid using bacteria of the genus Acetobacter, there are also a few processes for the preparation of a few carboxylic acids using bacteria of the genera Acetobacter or Gluconobacter or using yeasts.

For example, DE 3,713,668 describes the preparation of aliphatic carboxylic acids by microbial oxidation of aliphatic alcohols with bacteria of the species Gluconobacter roseus. In this process, the alcohols, after a growth phase of more than 24 hours, were added directly to the culture medium with the organism. The preferred pH range was stated as 4 to 4.5. Only low yields of 13 g of n-butyric acid/l 2 g of isobutyric acid/l, 7 g of 2-methylbutyric acid/l and 17 g of 3-methylbutyric acid/l of fermentation solution were obtained.

DE 19 503 598 describes a process for the preparation of propionic acid or butyric acid and salts thereof. They use a bacterium of the species Gluconobacter oxydans. After cultivation for 9 to 10 hours, n-propanol or n-butanol was repeatedly added in portions as a function of the pO₂ value. In this way they achieved yields of 43.7 g/l of propionic acid and 49 g/l of butyric acid.

EP 0 563 346 describes a process for the preparation of carboxylic acids by oxidizing corresponding alcohols or aldehydes using a yeast of the genus Saccharomyces, Hansenula, Pichia, Candida or Kluyvermyces. A disadvantage in this respect is that, using the yeasts, only low product concentrations are obtained, very high biomass concentrations have to be used and long process times. For example, after four days, only less than 0.6 g/l of 3-methylthiopropanolic acid was obtained, and for the 90% conversion of 0.01% of isoamyl alcohol, 6 days were needed.

J. Chem. Tech. Biotechnol. 1997, 68, 214-218 describes the biotransformation of a few aliphatic alcohols and 2-phenylethanol into the corresponding acids using bacteria of the species Acetobacter aceti. A disadvantage here, too, are the low product concentrations obtained. For example, the highest product concentration described for the oxidation of butanol to butyric acid was given as 39.3 g/l after 60 hours.

J. Chem. Tech. Biotechnol. 1997, 70, 294-298 describes the bacterium Acetobacter pasteurianus for the oxidative preparation of certain carboxylic acids. A disadvantage in this respect is the use of air-lift bioreactors since the high stream of air which is required for aerating and thoroughly mixing the culture causes relatively large amounts of the volatile starting materials and products to be stripped off. The cold trap containing liquid nitrogen, which is connected downstream for this reason, is not practicable on an industrial scale.

### SUMMARY OF THE INVENTION

We have found a process for the preparation of aliphatic, aromatic and thiocarboxylic acids in bioreactors, which is characterized in that cultures comprising bacteria of the genus Gluconobacter are used.

Surprisingly, the use of the novel organisms of the genus Gluconobacter enables very high yields not only of aliphatic, but also of aromatic and thiocarboxylic acids to be achieved. This is true both with regard to the product concentration in the solution, the percentage molar conversion of the starting materials, and also with regard to the space-time yield. Here, as well as the composition of the media and the pH, which is maintained at pH 6.4, a parameter which is of particular importance for the process is the nature of the continuous addition of the substrate.

### DETAILED DESCRIPTION OF THE INVENTION

Preferred bacteria for the process according to the present invention are bacteria of the type Gluconobacter sp. HR 101 (DSM 12884).

Preference is given to using the bacterium in pure culture.

Suitable nutrient media for the organisms used according to the present invention are synthetic, semisynthetic or complex culture media. These can comprise carbon-containing and nitrogen-containing compounds, inorganic salts, and optionally, trace elements and vitamins.

Carbon-containing compounds which may be suitable are carbohydrates, hydrocarbons or standard organic chemicals. Examples of compounds which may preferably be used are sugars, alcohols or sugar alcohols, organic acids or complex mixtures.

The sugar is preferably glucose. The organic acids which may preferably be used are citric acid or acetic acid. The complex mixtures include, for example, malt extract, yeast extract, casein or casein hydrolyzate.

Suitable nitrogen-containing substrates are inorganic compounds. Examples thereof are nitrates and ammonium salts. Organic nitrogen sources can also be used. These include yeast extract, soybean flour, casein, cottonseed meal, casein hydrolyzate, wheat gluten and corn steep liquor.

Examples of the inorganic salts which can be used are sulfates, nitrates, chlorides, carbonates and phosphates. The metals which are preferably present in said salts are sodium, potassium, magnesium, manganese, calcium, zinc and iron.

The cultivation temperature is preferably in the range from 10 to 40° C. The range is more preferably from 20 to 35° C.

The pH of the medium is preferably 4 to 8. A more preferred range is from 6.2 to 6.5.

In principle, all bioreactors known to the person skilled in the art can be used for carrying out the process according to the present invention. Preferential consideration is given to any equipment which is suitable for submerged processes. This means, according to the present invention, that it is possible to use vessels with or without a mechanical mixing device. Examples of the latter include shaking apparatuses, and bubble column reactors or loop reactors. The former preferably include all known appliances which are fitted with stirrers of any design.

The process according to the present invention can be carried out continuously or batchwise. The fermentation time required to achieve a maximum amount of product depends on the specific nature of the organism used. However, in principle, the fermentation times are between 2 and 200 hours.

Aliphatic carboxylic acids for the process according to the present invention are butyric acid, isobutyric acid, isovaleric acid, 2-methylbutyric acid and propionic acid.

Aromatic carboxylic acids for the process according to the present invention are benzoic acid, phenylacetic acid and cinnamic acid.

A thiocarboxylic acid for the process according to the present invention is 3-methylthiopropionic acid.

According to the process of the invention, preference is given to reacting butyric acid, isobutyric acid, isovaleric acid, 2-methylbutyric acid, propionic acid, phenylacetic acid and 3-methylthiopropionic acid.

According to the process of the invention, particular preference is given to reacting isobutyric acid, isovaleric acid, 2-methylbutyric acid and phenylacetic acid.

The invention is illustrated in more detail below by reference to examples:

### EXAMPLES

### Example 1

### Preparation of the Preculture

A 500 ml Erlenmeyer flask with a baffle is inoculated with 100 ml of a sterile medium consisting of 1.25 g of D-mannitol and 0.75 g of yeast extract at pH 6.5, with 0.9 ml of a glycerol culture of Gluconobacter sp. HR 101 (DSM 12884). The flask is incubated for 16 hours on a rotary shaker at 30° C. and 140 rpm. The number of microbes in the preculture is about 2×10⁹ CFU/ml.

### Example 2

### Preparation of Natural n-butyric Acid from Natural n-butanol

125 g of mannitol and 75 g of yeast extract are dissolved in 9.9 l of water in a 10 l fermenter, 10 ml of antifoam are added and the pH is adjusted to 6.3. The thus prepared medium is sterilized for 30 minutes at 121° C.

The speed of the stirrer is 500 rpm, and the aeration is 5 l/mm; the temperature is 30° C. After these parameters have been set, the preculture according to Example 1 is used for the inoculation.

After a fermentation time of 17 hours the addition of n-butanol is started via a pump. The metered addition of the substrate is controlled via a flow controller. n-Butanol is added in accordance with the following flow profile:

TABLE 1

| Fermentation | | | |
|---|---|---|---|
| time | | Flow rate | |
| 0 | h | 0 | g/lh |
| 17 | h | 1.0 | g/lh |
| 20 | h | 4.0 | g/lh |
| 27 | h | 3.0 | gIlh |
| 30 | h | 2.5 | g/lh |
| 35 | h | 2.0 | g/lh |
| 50 | h | 1.5 | g/lh |
| 50.5 | h | 2.0 | g/lh |
| 53 | h | 1.5 | g/lh |
| 57 | h | 1.0 | g/lh |
| 63 | h | 0 | g/lh |
| 66 | h | 1.0 | g/lh |
| 68 | h | 1.5 | g/lh |
| 71 | h | 1.0 | g/lh |
| 73 | h | 0 | g/lh |

During feeding, the pH is kept constant in the range 6.2-6.4 using NH₄⁺.

The fermentation is complete after 74 hours. The final concentration of n-butyric acid is 95 g/l according to HPLC analysis. The molar conversion is just below 90%.

### Example 3

### Preparation of Natural Isobutyric Acid from Natural Isobutanol

125 g of mannitol and 75 g of yeast extract are dissolved in 9.9 l of water in a 10 l fermenter, 10 ml of antifoam are added and the pH is adjusted to 6.3. The thus prepared medium is sterilized for 30 minutes at 121° C.

The speed of the stirrer is 500 rpm, and the aeration is 5 l/min; the temperature is 30° C. After these parameters have been set, the preculture according to Example 1 is used for the inoculation.

After a fermentation time of 22.5 hours the addition of isobutanol is started via a pump. The metered addition of the substrate is controlled via a flow controller. Isobutanol is added in accordance with the following flow profile:

TABLE 2

| Fermentation | | | |
|---|---|---|---|
| time | | Flow rate | |
| 0 | h | 0 | g/lh |
| 22.5 | h | 1.0 | g/lh |
| 23.5 | h | 4.0 | g/lh |
| 30 | h | 3.0 | g/lh |
| 35 | h | 2.5 | g/lh |
| 50 | h | 2.0 | g/lh |
| 50.3 | h | 2.5 | g/lh |
| 53 | h | 1.5 | g/lh |
| 58 | h | 1.0 | g/lh |
| 60 | h | 0 | g/lh |
| 64 | h | 1.0 | g/lh |
| 67 | h | 0 | g/lh |
| 68 | h | 1.5 | g/lh |
| 73 | h | 1.0 | g/lh |
| 74 | h | 0 | g/lh |

During the feeding, the pH is kept constant in the range 6.2-6.4 using NH₄⁺.

The fermentation is complete after 74 hours. The final concentration of isobutyric acid is 92.7 g/l according to HPLC analysis. The molar conversion is just below 88%.

### Example 4

### Preparation of Natural 2-methylbutyric Acid from Natural 2-methylbutanol

125 g of mannitol and 75 g of yeast extract are dissolved in 9.9 l of water in a 10 l fermenter, 10 ml of antifoam are added and the pH is adjusted to 6.3. The thus prepared medium is sterilized for 30 minutes at 121° C.

The speed of the stirrer is 500 rpm, and the aeration is 5 l/min; the temperature is 30° C. After these parameters have been set, the preculture according to Example 1 is used for the inoculation.

After a fermentation time of 17 hours the addition of 2-methylbutanol is started via a pump. The metered addition of the substrate is controlled via a flow controller. 2-Methylbutanol is added in accordance with the following flow profile:

TABLE 3

| Fermentation | | | |
|---|---|---|---|
| time | | Flow rate | |
| 0 | h | 0 | g/lh |
| 17 | h | 1.0 | g/lh |
| 20 | h | 4.0 | g/lh |
| 28 | h | 3.5 | g/lh |
| 31 | h | 3.0 | g/lh |
| 35 | h | 2.5 | g/lh |
| 39 | h | 2.0 | g/lh |
| 45 | h | 1.5 | g/lh |
| 51 | h | 1.0 | g/lh |
| 55 | h | 0 | g/lh |

During the feeding, the pH is kept constant in the range 6.2-6.4 using NH₄⁺.

The final concentration of 2-methylbutyric acid is 80 g/l according to HPLC analysis. The molar conversion is just below 89%.

### Example 5

### Preparation of Natural Isovaleric Acid from Natural Isoamyl Alcohol

125 g of mannitol and 75 g of yeast extract are dissolved in 9.9 l of water in a 10 l fermenter, 10 ml of antifoam are added and the pH is adjusted to 6.3. The thus prepared medium is sterilized for 30 minutes at 121° C.

The speed of the stirrer is 500 rpm, and the aeration is 5 l/min; the temperature is 30° C. After these parameters have been set, the preculture according to Example 1 is used for the inoculation.

After a fermentation time of 17 hours the addition of isoamyl alcohol is started via a pump. The metered addition of the substrate is controlled via a flow controller. Iso-amylalcohol is added in accordance with the following flow profile:

TABLE 4

| Fermentation | | | |
|---|---|---|---|
| time | | Flow rate | |
| 0 | h | 0 | g/lh |
| 17 | h | 1.0 | g/lh |
| 20 | h | 4.0 | g/lh |
| 28 | h | 3.5 | g/lh |
| 31 | h | 3.0 | g/lh |
| 35 | h | 2.5 | g/lh |
| 39 | h | 2.0 | g/lh |
| 44 | h | 1.5 | g/lh |
| 48 | h | 1.0 | g/lh |
| 49.5 | h | 0 | g/lh |
| 55 | h | 1.0 | g/lh |
| 58 | h | 0 | g/lh |
| 63 | h | 1.0 | g/lh |
| 66 | h | 0 | g/lh |

During the feeding, the pH is kept constant in the range 6.2-6.4 using NH₄⁺.

The fermentation is complete after 70.5 hours. The final concentration of isovaleric acid is 82 g/l following work-up of the fermentation solution. The molar conversion is just below 85%.

### Example 6

### Preparation of Natural Propionic Acid from Natural n-propanol

125 g of mannitol and 75 g of yeast extract are dissolved in 9.9 l of water in a 10 l fermenter, 10 ml of antifoam are added and the pH is adjusted to 6.3. The thus prepared medium is sterilized for 30 minutes at 121° C.

The speed of the stirrer is 500 rpm, and the aeration is 5 l/min; the temperature is 30° C. After these parameters have been set, the preculture according to Example 1 is used for the inoculation.

After a fermentation time of 17 hours the addition of propanol is started via a pump. The metered addition of the substrate is controlled via a flow controller. Propanol is added in accordance with the following flow profile:

TABLE 5

| Fermentation | | | |
|---|---|---|---|
| time | | Flow rate | |
| 0 | h | 0 | g/lh |
| 17 | h | 1.0 | g/lh |
| 20 | h | 3.5 | g/lh |
| 27 | h | 3.0 | g/lh |
| 29 | h | 2.0 | g/lh |
| 35 | h | 1.5 | g/lh |
| 60 | h | 1.0 | g/lh |
| 82 | h | 0 | g/lh |
| 87 | h | 1.0 | g/lh |
| 90 | h | 0 | g/lh |

During the feeding, the pH is kept constant in the range 6.2-6.4 using NH₄⁺.

The fermentation is complete after 92 hours. The final concentration of propionic acid is 94 g/l according to HPLC analysis. The molar conversion is 88.3%.

### Example 7

### Preparation of Natural Phenylacetic Acid from Natural Phenylethanol

125 g of mannitol and 75 g of yeast extract are dissolved in 9.9 l of water in a 10 l fermenter, 10 ml of antifoam are added and the pH is adjusted to 6.3. The thus prepared medium is sterilized for 30 minutes at 121° C.

The speed of the stirrer is 500 rpm, and the aeration is 5 l/min; the temperature is 30° C. After these parameters have been set, the preculture according to Example 1 is used for the inoculation.

After a fermentation time of 17 hours the addition of phenylethyl alcohol is started via a pump. The metered addition of the substrate is controlled via a flow controller. Phenylethyl alcohol is added in accordance with the following flow profile:

TABLE 6

| Fermentation | | | |
|---|---|---|---|
| time | | Flow rate | |
| 0 | h | 0 | g/lh |
| 17 | h | 1.0 | g/lh |
| 20 | h | 4.0 | g/lh |
| 23.5 | h | 2.0 | g/Ih |
| 24 | h | 2.5 | g/lh |
| 30 | h | 2.0 | g/lh |
| 37 | h | 1.5 | g/lh |
| 41 | h | 1.0 | g/lh |
| 43.8 | h | 0 | g/lh |
| 50.5 | h | 1.0 | g/lh |
| 53 | h | 0 | g/lh |
| 58 | h | 1.0 | g/lh |
| 60 | h | 0 | g/lh |
| 65 | h | 1.0 | g/lh |
| 67 | h | 0 | g/lh |

During the feeding, the pH is kept constant in the range 6.2-6.4 using NH₄⁺.

The maximum product concentration is reached after 48 hours. The concentration of phenylacetic acid is 54 g/l according to HPLC analysis. The molar conversion is 88.5%.

Transferring the process to the 200 l scale gave 52 g/l; the molar conversion in this case was 95%.

### Example 8

### Preparation of Natural Benzoic Acid from Benzyl Alcohol

125 g of mannitol and 75 g of yeast extract are dissolved in 9.9 l of water in a 10 l fermenter, 10 ml of antifoam are added and the pH is adjusted to 6.3. The thus prepared medium is sterilized for 30 minutes at 121° C.

The speed of the stirrer is 500 rpm, and the aeration is 5 l/min; the temperature is 30° C. After these parameters have been set, the preculture according to Example 1 is used for the inoculation.

After a fermentation time of 21.25 hours the addition of benzyl alcohol is started via a pump. The metered addition of the substrate is controlled via a flow controller. Benzyl alcohol is added in accordance with the following flow profile:

TABLE 7

| Fermentation | | | |
|---|---|---|---|
| time | | Flow rate | |
| 0 | h | 0 | g/lh |
| 21.25 | h | 1.0 | g/lh |
| 23 | h | 3.0 | g/lh |
| 28 | h | 2.5 | g/lh |
| 31 | h | 2.0 | g/lh |
| 34 | h | 1.5 | g/lh |
| 37 | h | 1.0 | g/lh |
| 40 | h | 0 | g/lh |
| 43 | h | 1.0 | g/lh |
| 47.5 | h | 0 | g/lh |
| 50.5 | h | 1.0 | g/lh |
| 54 | h | 0 | g/lh |
| 57 | h | 1.0 | g/lh |
| 60 | h | 0 | g/lh |
| 63 | h | 1.0 | g/lh |
| 65 | h | 0 | g/lh |

During the feeding, the pH is kept constant in the range 6.2-6.4 using NH₄⁺.

The fermentation is complete after 68 hours. The final concentration of benzoic acid is 51 g/l according to HPLC analysis. Virtually all of the starting material was converted.

### Example 9

### Preparation of Natural Cinnamic Acid from Cinnamyl Alcohol

125 g of mannitol and 75 g of yeast extract are dissolved in 9.9 l of water in a 10 l fermenter, 10 ml of antifoam are added and the pH is adjusted to 6.3. The thus prepared medium is sterilized for 30 minutes at 121° C.

The speed of the stirrer is 500 rpm, and the aeration is 5 l/min; the temperature is 30° C. After these parameters have been set, the preculture according to Example 1 is used for the inoculation.

After a fermentation time of 17 hours, the addition of cinnamyl alcohol is started via a pump. In order to have the cinnamyl alcohol in the liquid phase, the starting material was heated. Cinnamyl alcohol is added according to the following flow profile:

TABLE 8

| Fermentation | | | |
|---|---|---|---|
| time | | Flow rate | |
| 0 | h | 0 | g/lh |
| 17 | h | 1.2 | g/lh |
| 20 | h | 2.4 | g/lh |
| 21 | h | 3.6 | g/lh |
| 25.25 | h | 0 | g/lh |
| 26.25 | h | 2.4 | g/lh |
| 28 | h | 1.2 | g/lh |
| 30 | h | 0 | g/lh |
| 31 | h | 1.2 | g/lh |
| 32 | h | 0 | g/lh |

The fermentation is complete after 44 hours. The final concentration of cinnamic acid is 27 g/l according to HPLC analysis. Virtually all of the starting material was converted.

### Example 10

### Preparation of Natural 3-methylthiopropionic Acid from 3-methylthiopropanol

125 g of mannitol and 125 g of yeast extract are dissolved in 10 l of water in a 10 l fermenter, 10 ml of antifoam are added and the pH is adjusted to 6.3. The thus prepared medium is sterilized for 30 minutes at 121° C.

The speed of the stirrer is 500 rpm, and the aeration is 5 l/min; the temperature is 27° C. After these parameters have been set, the preculture according to Example 1 is used for the inoculation.

After a fermentation time of 16 hours, the addition of 3-methylthiopropanol is started via a pump. The substrate is metered in accordance with the following flow profile:

TABLE 9

| Fermentation | | | |
|---|---|---|---|
| time | | Flow rate | |
| 0 | h | 0 | g/lh |
| 16 | h | 1.0 | g/lh |
| 17.66 | h | 2.0 | g/lh |
| 19 | h | 3.0 | g/lh |
| 20.8 | h | 4.2 | g/lh |
| 21.8 | h | 4.8 | g/lh |
| 23.2 | h | 4.2 | g/lh |
| 23.8 | h | 2.6 | g/lh |
| 42.5 | h | 2.2 | g/lh |
| 48.8 | h | 0 | g/lh |

During the feeding, the pH is kept constant in the range 6.2-6.4 using NH₄⁺.

The fermentation is complete after 65 hours. The final concentration Of 3-methylthiopropionic acid is 82.6 g/l according to HPLC analysis. The molar conversion is almost 100%.

### Example 12

### Comparison of the Space-Time Yield of the Present Invention with Those of the Known Processes

In the example below (Table 9), the space-time yields of the novel production process using Gluconobacter sp. DSM 12884 are compared with those of the known processes in order to demonstrate the superiority of the process of the present invention.

TABLE 10

| | | | Product | | |
|---|---|---|---|---|---|
| Test | | Process time | concentration | Space-time | |
| Number | Product | [hours] | [g/l] | yield [g/l/h] | Process |
| 1 | Butyric acid | 120 | 13 | 0.11 | DE-A-37 13 668 |
| 2 | Butyric acid | 60 | 39.3 | 0.66 | J. Chem. Tech. Biotechnol. 1997, 68, 214-218 |
| 3 | Butyric acid | 80 | 49 | 0.61 | DE-A-195 03 598 |
| 4 | Buryric acid | 90 | 60 | 0.67 | J. Chem. Tech. Biotechnol. 1997, 70, 294-298 |
| 5 | Buryric acid | 73 | 95 | 1.3 | Present invention |
| 6 | Propionic acid | 70 | 43.7 | 0.62 | DE-A-1 95 03 598 |
| 7 | Propionic acid | 90 | 60 | 0.67 | J. Chem. Tech. Biotechnol. 1997, 70, 294-298 |
| 8 | Propionic acid | 92 | 94 | 1.02 | Present invention |
| 9 | Isobutyric acid | 42 | 21 | 0.5 | DE-A-37 13 668 |
| 10 | Isobutyric acid | 74 | 92.7 | 1.25 | Present invention |
| 11 | Isovaleric acid | 25 | 17 | 0.68 | DE-A-37 13 668 |
| 12 | Isovaleric acid | 90 | 45 | 0.5 | J. Chem. Tech. Biotechnol. 1997, 70, 294-298 |
| 13 | Isovaleric acid | 70.5 | 82 | 1.16 | Present invention |
| 14 | 2-Methylbutyric acid | 24 | 7 | 0.29 | DE-A-37 13 668 |
| 15 | 2-Methylbutyric acid | 90 | 44 | 0.49 | J. Chem. Tech. Biotechnol. 1997, 70, 294-298 |
| 16 | 2-Methylburyric acid | 52 | 80 | 1.54 | Present invention |

As Table 10 shows, use of the present invention with Gluconobacter sp. DSM 12884 in the already-described alcohol oxidations results in a large increase in both the space-time yield and the absolute product concentration. For example, in the case of butyric acid, the space-time yield increases by 94% and the product concentration by 58%, when the present invention (Test No. 5) is compared with the best result from the prior art (Test No. 4). In the case of propionic acid, the space-time yield increases by 52% and the product concentration by 57%. The increases in the case of isobutyric acid are even greater, being 150% for the space-time yield and 341% for the product concentration. The increases for isovaleric acid and 2-methylbutyric acid are 132% and 82%, and 214% and 82% respectively.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. Process for the preparation of aliphatic, aromatic and thiocarboxylic acids in bioreactors, characterized in that cultures comprising bacteria of the genus Gluconobacter are used.

2. Process according to claim 1, characterized in that a pure culture consisting of bacteria of the genus Gluconobacter is used.

3. Process according to one of claims 1 or 2, characterized in that Gluconobacter sp. HR 101 (DSM 12884) is used.

4. Process according to one of claims 1 to 3, characterized in that synthetic, semisynthetic or complex substrates are used.

5. Process according to one of claims 1 to 4, characterized in that substrates comprising carbon-containing and nitrogen-containing compounds, inorganic salts, trace elements and/or vitamins are used.

6. Process according to claim 5, characterized in that the substrate comprises, as carbon-containing compounds, sugar, sugar alcohols, alcohol, organic acids, complex mixtures, oils or mixtures of two or more of these substances.

7. Process according to claim 6, characterized in that substrates comprising glucose, glycerol, mannitol, citric acid, malt extract, yeast extract, casein, casein hydrolysate and castor oil or mixtures of two or more of these substances are used.

8. Process according to one of claims 1 to 7, characterized in that substrates comprising inorganic compounds and/or organic compounds are used.

9. Process according to claim 8, characterized in that nitrates, ammonium salts, yeast extract, soybean flour, cottonseed meal, casein, casein hydrolysate, wheat gluten and maize steep liquor are used.

10. Process according to one of claims 1 to 9, characterized in that substrates comprising sulphates, nitrates, chlorides, carbonates and phosphates of the metals sodium, potassium, magnesium, manganese, calcium, zinc and iron or mixtures of two or more of these compounds are used.

11. Process according to one of claims 1 to 10, characterized in that the temperatures are in the range from 10 to 40° C.

12. Process according to one of claims 1 to 11, characterized in that the pH is in the range from 4 to 8.

13. Gluconobacter sp. HR 101 (DSM 12884).

14. Use of Gluconobacter sp. according to claim 13 for the preparation of aliphatic, aromatic and thiocarboxylic acids.

15. Process for the preparation of aliphatic, aromatic and thiocarboxylic acids, characterized in that the molar conversion of the starting materials is greater than 60%.

16. Process for the preparation of natural butyric acid in bioreactors, characterized in that cultures comprising bacteria of the genus Gluconobacter are used.

17. Process for the preparation of isobutyric acid in bioreactors, characterized in that cultures comprising bacteria of the genus Gluconobacter are used.

18. Process according to claim 16 and 17, characterized in that a pure culture consisting of bacteria of the genus Gluconobacter is used.

19. Process according to claim 16, 17 and 18, characterized in that Gluconobacter sp. HR 101 (DSM 12884) is used.
